(19) United States
(12) Patent Application Publication
(10) Pub. No.: US 2007/0003487 A1
(43) Pub. Date: Jan. 4, 2007

(54) Composition, method and pharmaceutical preparation for pharmaceutical spray suspensions

(76) Inventor: Ragnar Ek, Stockholm (SE)

Correspondence Address: YOUNG & THOMPSON, 745 SOUTH 23RD STREET, 2ND FLOOR, ARLINGTON, VA, 22202, US

(21) Appl. No.: 10/553,389

(22) PCT Filed: Apr. 16, 2004
(86) PCT No.: PCT/SE04/00591

§ 371 Date: Apr. 11, 2006

Related U.S. Application Data
(60) Provisional application No. 60/463,326, filed on Apr. 17, 2003.

(51) Int. Cl. A61K 9/14; A61K 31/485; A61K 31/24; A61K 31/192; A61L 9/04

(52) U.S. Cl. 424/46; 514/282; 514/571; 514/536

(57) ABSTRACT

A pharmaceutical composition, constituting a spray suspension includes at least one liquid excipient and at least one solid excipient substantially insoluble in the liquid excipient, and at least one pharmaceutical active ingredient. A method of preparing porous suspension particles includes: a) wet-milling or dry-milling the solid excipient(s) or a mixture of at least one active ingredient and a solid excipient(s) in a milling equipment inducing essentially compression and shear forces, resulting in fine particulate quality, where more than 90% by weight is smaller than 5 m; b) drying and aggregating the product of step a) alone or with the addition of at least one active ingredient, in fine particulate form, which will produce essentially isodiametrical aggregate particles. A suspension particles obtainable by the method pharmaceutical preparation, utilising the composition or porous suspension particles and a method for treatment of disorders using the preparation are disclosed.

### FIELD OF INVENTION

The present invention relates to a pharmaceutical composition for administering drugs by spraying, to a method for preparing such a composition, to pharmaceutical preparations utilising the composition and to a method for the treatment of disorders by the use of such a composition. The present invention is primarily intended for transdermal (cutaneous) administration but can also be used for nasal administration or administration to the ear.

### BACKGROUND OF THE INVENTION

The common way to apply drugs topically (by the transdermal route) is by using ointments, creams, gels or patches. Transdermal sprays (cutaneous sprays) are however less frequently used.

In textbooks of pharmaceutics, two advantages are often mentioned for using a transdermal spray. Firstly, the drug is applied in a convenient manner, and secondly, sterility can more easily be maintained. In wound care it is preferable to avoid direct contact with the wound. When using sprays, the drug is applied without direct contact, while ointments and creams are applied through direct contact, i.e. have to be smeared out. Of equal importance is an easy removal after that the drug has lost its effect. This is not always possible with ointments, gels and creams since the excipients often are tacky, fat and viscous and therefore difficult to remove. Further, patches are inflexible in size and difficult to handle around joints. In spite of the advantages of cutaneous sprays in comparison with other transdermal dosage forms, there are only a minute number of cutaneous spray products on the market.

In principal there are three types of cutaneous sprays, that might be considered for drug administration. Firstly, there are spray solutions, where the active ingredient is molecularly dissolved in a liquid. A second alternative is the use of spray powders, where no liquid phase is present. Thirdly, the active ingredient could be dispersed in a liquid in the form of drug particles, forming a spray suspension. This type of cutaneous spray, i.e. spray suspensions, is seldom used for drug administration.

Spray solutions show potential problems regarding chemical stability of the solved drug and difficulties in regulating the drug release rate.

These difficulties can be solved if the drug is suspended and not dissolved in the liquid phase of the spray preparation. A drug in suspended form will by definition be more chemically stable. Also the possibility to retard the drug release might be improved by using a suspension form. Normally, the intact, healthy skin is a tight barrier to most drugs and therefor represents the rate limiting step regarding absorption. But for injured or inflamed skin, with loss of stratum corneum and altered keratinization, the permeability increases. In these situations it might be of special importance with a spray capable of sustaining drug release.

Although a spray suspension in principle can be formulated to give a cutaneous product with improved chemical stability and also facilitate the possibility to regulate the drug release rate, this approach is seldom utilised. The explanation is probably that drugs in suspended form are known to possess some principal drawbacks such as crystal growth and particle sedimentation leading to caking. Further, if a suspension is actuated with a spray bottle, it is believed, that the spray nozzle easily can be clogged. In the field where spray suspensions are frequently used, i.e. inhalation therapy, these drawbacks are generally solved by using the drug in micronised form (i.e. very fine particulate form).

Such type of very fine particulate suspension, in inhalation therapy, is of course also necessary in order to obtain an effective drug retention in the lung alveols. Using coarser drug particles will for inhalation preparations result in a deposition in the upper respiratory tract resulting in a rapid clearance from the lung, whereby the therapeutic effect will be missing.

The application of micronised drug suspensions for cutaneous spraying is in practice very difficult. In order to use such fine particulate drug qualities it is necessary to add one or several excipients to avoid aggregation and flocculation of the drug particles, in order to avoid sedimentation and caking. Such excipients are chosen from the groups of surfactants and/or electrolytes. These groups of excipients will inherently lead to irritation in injured or inflamed skin. Further, the use of micronised drug qualities will also limit the dosages that can be administered but it will also constitute a limitation with regards to the possibility to achieve an extended drug release. Even very sparingly soluble drugs will then, due to their small particle size, be dissolved almost instantly, thereby counteracting a retarded drug absorption.

It is therefore obvious that there is a need for improved cutaneous spray preparations where the active ingredient is present in a form improving stability but where also the drug release can be retarded to give an extended duration of the effect.

### SUMMARY OF THE INVENTION

It has now, surprisingly, been discovered that the addition of a solid excipient can solve the actual problems. The use of suspended solid excipients in cutaneous sprays is not previously described. Such an addition is regarded as not only unnecessary but also as an obstacle in obtaining a technically robust formulation. The use of solid, dispersed excipients can be used in two different ways to obtain the advantages described by the new invention. These two approaches will now be briefly described under separate headings.

The present invention thus solves the above problems by providing, according to a first aspect a pharmaceutical composition, constituting a spray suspension comprising at least one liquid excipient and at least one solid excipient which essentially is insoluble in the liquid excipient, and at least one pharmaceutical active ingredient. According to a second aspect of the present invention, there is provided a method of preparing porous suspension particles (comprising an active ingredient), wherein it comprises the steps of;
a. wet-milling or dry-milling the solid excipient(s) or a mixture of at least one active ingredient and a solid excipient(s) in a milling equipment inducing essentially compression and shear forces, resulting in fine particulate quality, where more than 90% by weight is smaller than 5 μm and preferably smaller than 2 μm; and
b. drying and aggregating the product of step a. or the product of step a. with the addition of at least one active ingredient, in fine particulate form, by e.g. spray-drying or any other drying procedure possible, which will produce essentially isodiametrical aggregate particles.

According to a third aspect of the present invention, there is provided a suspension particles obtainable by a method according to the second aspect. According to a fourth aspect of the present invention, there is provided a pharmaceutical preparation, utilising the composition according to the first aspect or porous suspension particles according to the third aspect wherein the preparation is a cutaneous spray, an ear spray or a nasal spray. According to a fifth aspect of the present invention there is provided a method for treatment of disorders, wherein an individual afflicted with disorder is administered a pharmaceutical composition, constituting a spray suspension comprising at least one liquid excipient and one solid excipient which essentially is insoluble in the liquid excipient and at least one pharmaceutical active ingredient.

The Use of Solid, Dispersed Excipients to Form a Coherent Porous Matrix, In Situ, on Skin.

Firstly the solid excipient can be used in relatively moderate particle sizes (normally smaller than 25-50 μm), dispersed in the liquid in which the drug is either molecularly dissolved or present in fine particulate or micronised form. During administration of such a product, a matrix will gradually be formed onto the skin, where the thickness of the matrix is determined by the time length of the spray actuation. This matrix could also be called a layer or a coat. Irrespective of which term is used the matrix consists of a network of excipient particles. In the inter particulate pores or voids, the drug substance will be present in particulate form or partly dissolved form. In the case where the entire amount of drug or a fraction of drug is initially, molecularly dissolved in the spray liquid, the extent or fraction of dissolved versus re-crystallised drug in the matrix porous system is dependent on the rate of evaporation of the spray liquid. The thickness of the matrix is normally at least 5-20 times the average diameter of the excipient particles, thus forming a truly porous structure within the matrix. By this embodiment of the invention a drug containing matrix can be formed in-situ on the skin, thereby permitting both a relatively rapid release, but also the basis for an extended release preparation.

Irrespective of how the drug is present in the matrix formed onto the skin, the addition of insoluble excipient particles in the spray preparation brings the following advantages to the cutaneous spray preparation in comparison with spray solutions and micronised spray suspensions (normally used for inhalation systems).
1. The matrix formed in-situ on the skin can result in administration of higher dosages and rate retarding properties regarding drug release.
2. The matrix formed in-situ on the skin can result in a more controlled drug re-crystallisation within the matrix for subsequent controlled drug release.
3. The matrix formed in-situ on the skin can result in the possibility to formulate spray preparations that easily adhere to the skin (bioadhesive properties) at the same time as they can easily be rinsed away.
   The Use of Relatively Large Spray Particles Formed Both of an Insoluble Excipient and Drug.

In this second approach of the new invention the suspension particles (the particles finally suspended in liquid phase of the product) should be composed of both a solid excipient(s), essentially insoluble in the liquid and also at least one active ingredient. The term suspension particles will hereafter frequently be used (especially in the claims 11-20) to denote such particles composed of both excipient and drug, supended in the spray liquid, irrespective of whether the liquid is a pressurised gas (propellant) or it is a liquid (such as water) at ambient conditions. In a preferred embodiment of the invention larger suspension particles are prepared by a size enlargement process where the solid excipient (fine particulate grade) and the active ingredient are co-processed to form the final suspension particles. The solid excipient can also be added in the form of relatively large, pre-formed, porous particles, into which the active ingredient has been incorporated thereby creating suspension particles. In both cases, such suspension particles have a porous structure where the solid excipient constitutes a matrix for the active ingredient. Thereby, it is possible to obtain a diffusional, controlled drug release out of the particle matrix.

In another embodiment of the invention, the solid excipient is in the form of relatively large, pre-formed, non-porous particles. In this case, the active ingredient is adhering to the surface of the solid excipient, thereby creating suspension particles. In this case, the use of an outer membrane (e.g. applied by a coating process) can be used for retarding the drug release.

Irrespective how the particles in the spray suspension are prepared, the addition of an insoluble excipient brings the following advantages to the cutaneous spray preparation in comparison with spray solutions and micronised spray suspensions (normally used for inhalation systems).
1. Large particles with well-defined pore structure and/or surface texture can be prepared, thus enabling administration of higher dosages and rate retarding properties regarding drug release.
2. Large particles with well-defined shape and surface texture can be prepared, thus avoiding clogging of the actuation nozzle.
3. Large particles can be prepared, where the size and shape essentially is determined by the solid excipient, thereby essentially limiting the effect of drug re-crystallisation and uncontrolled particle growth.
4. Large particles can be prepared, where the size and shape essentially is determined by the solid excipient, thereby making it possible to formulate suspensions that can be easily re-dispersed, i.e. caking is avoided.
5. Large particles can be prepared, where the bioadhesive properties essentially is determined by the solid excipient, thereby making it possible to formulate suspensions that easily adhere to the skin at the same time as they can easily be rinsed away.

### DETAILED DESCRIPTION OF THE INVENTION

Properties and Preparation of Spray Suspensions Intended to Form a Coherent, Porous Matrix, In Situ, on Skin

Important Aspects on the Matrix Forming Excipent Particles

Here, relatively fine particulate excipient particles, insoluble in the spray liquid are used. Preferably at least 90% by weight of these particles have a particle size less than 50 μm and at least 50% by weight have a particle size not less than 0.1 μm. These particles can be composed of e.g. starch, starch derivatives, celluloses and cellulose derivatives. In a preferred form microcrystalline celluloses in fine grade, such as Avicel PH 105 is used. Such excipient particles are dispersed in the spray liquid together with the active ingredient which can be dissolved or suspended in the liquid.

If the primary particle size of the excipient is low, the corresponding matrix formed on the skin will have a pore structure with a low average pore diameter. In principal, the lower the primary particle size is, the lower will the pore diameter be in the matrix. Thereby, the diffusion of the drug molecules within this matrix will not just be a simple diffusion transport in a liquid phase, but the diffusion will be significantly retarded by the pore structure.

An approach to further reduce the release rate is to fill the pores of the porous matrix by adding to the composition also a sparingly soluble excipient which has lipophilic properties or a high deformability, resulting in a relatively complete filling of the intra-particulate pores, thus further narrowing the effective pore diameter available for drug diffusional transport.

Important Aspects on How the Drug Release Rate and the Duration of Drug Release Can be Controlled Using Matrix Forming Excipent Particles

During the development of the present invention it was found that the amount of drug released per unit time (i.e. drug release rate) could be controlled by the surface area covered on skin after administration of a spray suspension. Then the drug release rate was directly proportional to the surface area of drug matrix applied on skin. The larger the surface area covered, the larger was the amount of drug released per unit time. One way to standardise or obtain a specific surface area of drug matrix covering the skin, was then to use e.g. a piece of paper with a circular hole with known diameter. If this paper first was put on the skin or in front of the skin, followed by spray administration, only the spray components passing the circular hole would reach the skin and subsequently form a circular/cylindrical drug matrix. The fraction of spray components impacting on the paper, instead of the hole, was consequently blocked out and not participating in the matrix formation on the skin. Thus by using a device with a range of increasingly sized openings or a device with a diaphragm where the opening diameter can be varied it is possible to control the drug release rate in connection with drug administration via a suspension spray.

It was also found that the matrix formed through a circular opening could be regarded as a matrix of cylindrical shape. Then, a prolongation of spraying will not alter the diameter of the cylindrical matrix but instead the height of the cylinder. It was then experienced that the longer the spraying time, the thicker the drug matrix and the longer the duration of drug release. It should be noted that such a prolongation of release duration will not affect the drug release rate. The rate of release will be controlled by the diameter, i.e. the surface area of the cylindrical drug matrix.

Thus by fine-tuning the surface area of skin covered (e.g. by using a device with defined openings) and the height of drug matrix (e.g. by using a specific spraying time), it is possible to obtain drug delivery system where both the drug release rate and release duration can be effectively controlled.

According to a preferred embodiment of the fifth aspect of the present invention there is accordingly provided a method for treatment of disorders wherein the drug release rate is controlled by varying the area of said composition covering the skin of an individual. Preferably the drug release rate is controlled by using a device with a range of increasingly sized openings or a device with a diaphragm where the opening diameter can be varied.

According to a preferred embodiment of the fifth aspect of the present invention there is also accordingly provided a method for treatment of disorders wherein the drug release duration is controlled by varying the height of said composition covering the skin of an individual. Preferably the drug release duration is controlled by using a specific spraying time.

According to a preferred embodiment of the fifth aspect of the present invention there is also accordingly additionally provided a method for treatment of disorders wherein the drug release rate is controlled by varying the area of said composition covering the skin of an individual, and wherein the drug release duration is controlled by varying the height of said composition covering the skin of an individual. Preferably the drug release rate is controlled by using a device with a range of increasingly sized openings or a device with a diaphragm where the opening diameter can be varied. Preferably the drug release duration is controlled by using a specific spraying time.

Properties and Preparation of Spray Suspensions Containing Relatively Large Suspension Particles

Important Aspects on the Use of Porous Suspension Particles

When the suspension particles, composed of both excipient(s) and drug component, are porous the drug release is determined by the diffusional transport out of the porous system. Since the particles in a spray suspension has to be rather small (here an average diameter of 50 μm is assumed), the drug molecule has a very short distance to diffuse before being released. The time t to diffuse a distance x of 25 μm can be estimated with Stoke-Einstein equation by assuming the diffusion coefficient for the drug to be 7·10⁻¹⁰ m²/s (a typical diffusion coefficient for a molecule with a molecular weight of 100 Dalton) and assuming that the intra particulate pores are relatively wide, thereby not hindering the diffusion transport;

$$t = \sqrt{\frac{{x}^{2}}{2 D}} = 0.67 s$$

The calculation reveals that the time for the drug to diffuse out of a particle is less than a second. Obviously there is a need to delay the drug release by means of some new barrier approach.

Although there has been shown that the diffusion coefficient for water decreases within a cellulose granule, it is not so pronounced that it can be used for sustaining the drug release¹. One new approach that has been discovered is to use extremely fine particulate grades of the solid excipient prior to the processing of the larger suspension particles. If the primary particle size of the excipient is low, preferably smaller than 2 μm, the corresponding large aggregate particles (either co-processed to contain an active ingredient or prepared to a porous excipient particle, subsequently filled with drug) will have a pore structure with a low average pore diameter. In principal, the lower the primary particle size is, the lower will the pore diameter be in the aggregate particles. Thereby, the diffusion of the drug molecules within these particles will not longer be a simple diffusion transport in a liquid phase, but the diffusion will be significantly retarded by the pore structure.
¹ Ek R., Lennholm H., Davidsson R., Nyström C. and Ragnarsson G. Pore swelling in beads made of cellulose fibres and fibre fragments. Int. J. Pharm. 122 (1995) 49-56.

It ought to be mentioned that the term porous, suspension particles here does not necessary imply that the porosity is high, but rather that there exist a certain amount of intraparticulate porosity into which the drug can be incorporated for subsequent release.

An approach to further reduce the release rate is to fill the pores of the porous suspension particles with a sparingly soluble excipient which has a high deformability, resulting in a relatively complete filling of the intra-particulate pores, thus further narrowing the effective pore diameter available for drug diffusional transport.

Preparation of Porous Suspension Particles

Based on Co-Processing of a Solid Excipient and an Active Ingredient

The active ingredient is firstly dry mixed with at least one solid excipient. This mixture is then milled in a suitable milling equipment to obtain a very fine particulate quality of the powder mixture. Alternatively, the solid excipient(s) are milled separately and subsequently admixed to the active ingredient, which already is present in a fine particulate grade. In a second step larger particles (thus containing at least one active ingredient as well as solid excipients) are manufactured in e.g. a spraydrier. The obtained particles should preferably have a diameter between 10 and 150 μm and more preferably around 50 μm. These particles are by definition aggregates of the small primary particles of drug and excipient. The pore system in the particles can be varied using various types of solid excipents and using various degrees of fineness of the solid excipient and active ingredient prior to the size enlargement process. The drug release will be related to the pore structure of the particles. It is thus possible to obtain e.g. a pronounced slow release profile by lowering the pore size down even to the nano size range.

Based on Large, Preformed, Porous, Solid Excipient Particles

Excipient particles are prepared, e.g. in accordance with the description above, with the exception that no drug is incorporated. After spray drying the empty porous excipient particles, a drug is incorporated via e.g. a sorption process. Then the drug solution or drug suspension is admixed with the porous excipient particles for a time period long enough to allow the drug to fill an adequate fraction of the pore volume of the excipient particles.

Preparation of Non-Porous Suspension Particles

Here, the use of commercially available excipient beads is preferred. These are normally composed of starches, celluloses or derivatives thereof. Also the use of inorganic salts, such as calcium carbonate, barium sulphate etc., can be considered. The drug component is then dissolved or dispersed in a liquid and coated onto the excipient beads by e.g. a process utilising a fluid bed equipment.

Important Aspects on the Use of Release Retarding Membranes

Another approach to reduce the release rate from spray suspension particles is to apply an outer membrane barrier by e.g. a coating process. Such membranes can be formed by a range of polymeric materials. However, also other membrane forming materials can be used, well-known to a person skilled in the art. This type of release retardation system can obviously be added to porous suspension particles to further reduce the drug release rate but can also be applied to suspension particles which contain the active ingredient adhering to an essentially non-porous solid excipient particle. Suitable excipient materials for forming coatings layers (or membranes) for delayed and/or extended release are non-polymeric- or polymeric materials such as calcium phosphate, ethyl cellulose, methacrylate copolymer, polyamide, polyethylene, polyvinyl alcohol or polyvinyl acetate.

Preparation of Spray Suspensions

Pressurised Aerosols

These can be prepared in accordance with what is described in textbooks such as “The theory and practice of industrial pharmacy”, 2^{nd} edition, Eds. Lachman, Lieberman and Kanig, Lea & Febiger, Philadelphia 1976, page 270-295, which is hereby incorporated as reference. Such dosage systems are well known to the persons skilled in the art. Here, the liquid used in the dosage form is pressurised gas. Due to the environmental impact, the freones, earlier used frequently as propellants are today largely exchanged with e.g. dimethyl ether and mixtures of propane/butane. It has been demonstrated during the work with this invention that to obtain improved functional behaviour of the aerosol, water should be added to the propellant. E.g. using dimethyl ether, it was shown that at least 10-95% of the composition should be water and more preferably at least 30% should be water.

Pump Aerosols

Alternatively the drug and excipient materials according to this invention could be filled in containers for manually pumping out liquid sprays. Then, the liquid is preferably water or mixtures of water and alcohols.

According to a further preferred embodiment of the first aspect of the present invention there is provided a pharmaceutical composition wherein the liquid excipient is a pressured aerosol propellant, such as dimethylether, butane, propane, mixtures of butane and propane, fluorinated hydro carbons, nitrogen, carbon dioxide and nitrous oxide.

According to a further preferred embodiment of the first aspect of the present invention there is provided a pharmaceutical composition wherein also water is included in the composition, preferably in a concentration between 10-95 w/w %, and more preferably in a concentration between 30-95%.

According to a further preferred embodiment of the first aspect of the present invention there is provided a pharmaceutical composition wherein the liquid excipient is water or a mixture of water and an organic solvent, such as alcohols.

According to a further preferred embodiment of the first aspect of the present invention there is provided a pharmaceutical composition wherein the solid excipent consists of inorganic salts or polymers selected from the group consisting of natural polymers, modified natural polymers, synthetic polymers and mixtures thereof.

According to a further preferred embodiment of the first aspect of the present invention there is provided a pharmaceutical composition wherein the polymeric material consists of natural polymers selected from the group consisting of native cellulose, such as Cellulose I.

According to a further preferred embodiment of the first aspect of the present invention there is provided a pharmaceutical composition wherein the native cellulose is micro crystalline cellulose or milled qualities of micro crystalline cellulose.

According to a further preferred embodiment of the first aspect of the present invention there is provided a pharmaceutical composition wherein the excipient particles are suspended in the liquid excipient, wherein the active ingredient is either dissolved, partly dissolved or suspended in the liquid or precipitated on the surface of the solid excipient and where the excipient particles after actuation can form a matrix, in-situ, on the administration site, such as the skin.

According to a further preferred embodiment of the first aspect of the present invention there is provided a pharmaceutical composition wherein the composition also contains at least one additional solid excipient which is capable of retarding the drug release from the matrix formed in-situ.

According to a further preferred embodiment of the first aspect of the present invention there is provided a pharmaceutical composition wherein at least 50% by weight of the excipient particles have a particle size not less than 0.1 μm and where at least 90% by weight of the excipient particles have a particle size less than 50 μm.

According to a further preferred embodiment of the first aspect of the present invention there is provided a pharmaceutical composition wherein the excipient particles together with the active ingredient forms a plurality of larger individual particles (suspension particles).

According to a further preferred embodiment of the first aspect of the present invention there is provided a pharmaceutical composition wherein the excipient particles together with the active ingredient forms a plurality of larger individual particles (suspension particles) that are porous and that the composition also contains at least one additional solid excipient which is capable of retarding the drug release from the suspension particles.

According to a further preferred embodiment of the first aspect of the present invention there is provided a pharmaceutical composition wherein the additional solid excipient is a polymer, with pronounced ductile properties thereby capable of reducing the porosity and/or average poor diameter of the suspension particles, or the matrix formed in-situ.

According to a further preferred embodiment of the first aspect of the present invention there is provided a pharmaceutical composition wherein the composition also contains at least one additional solid excipient which is capable of forming an outer membrane layer around the suspension particles, where the membrane layer retards the drug release and where the membrane layer is composed of non-polymeric- or polymeric materials such as calcium phosphate, ethyl cellulose, methacrylate copolymer, polyamide, polyethylene, polyvinyl alcohol or polyvinyl acetate.

According to a further preferred embodiment of the first aspect of the present invention there is provided a pharmaceutical composition wherein at least 50% by weight of the suspension particles have a particle size not less than 10 μm and where at least 90% by weight have a particle size smaller than 150 μm.

According to a further preferred embodiment of the first aspect of the present invention there is provided a pharmaceutical composition wherein the suspension particles have an essentially isodiametrical shape, and preferably the particles also have a smooth surface texture.

According to a further preferred embodiment of the second aspect of the present invention there is provided a method of preparing porous suspension particles (comprising an active ingredient), according to the first aspect of the present invention wherein it comprises the steps of;
a. porous excipient particles, excluding any active ingredient, (thus not including any active ingredient) are prepared in accordance with the method of the second aspect of the present invention; and
b. at least one active ingredient is added to the product of step a. whereby the active ingredient is essentially positioned within the pore structure of the product of step a.

According to a further preferred embodiment of the second aspect of the present invention there is provided a method of preparing non-porous suspension particles (including an active ingredient) wherein the active ingredient is applied, by e.g. a coating process, as an outer layer on solid, non-porous, excipient particles.

According to a further preferred embodiment of the second aspect of the present invention there is provided a method of applying a drug release retarding outer membrane layer to the suspension particles as set out above and where the membrane layer is composed of non-polymeric- or polymeric materials such as calcium phosphate, ethyl cellulose, methacrylate copolymer, polyamide, polyethylene, polyvinyl alcohol or polyvinyl acetate.

According to a further preferred embodiment of the fourth aspect of the present invention there is provided a pharmaceutical preparation, wherein the preparation contains as the active substance, morphine, morphine sulphate, morphine hydrochloride, ketoprofen or other substances effective in the treatment of pain or capable of inducing anestethic effect.

According to a further preferred embodiment of the fourth aspect of the present invention there is provided a pharmaceutical preparation wherein the preparation is in the form of a pressurised aerosol or mechanical pump device.

### EXAMPLES

### Example 1

Spray Composition Containing Smaller Excipient Suspension Particles for Forming a Coherent, Porous Matrix, In Situ, on Skin

300 g Microcrystalline cellulose (Avicel PH 105) was suspended into 690 g distilled water containing 10 g NaCl (used as a model drug substance). The mixture was homogenised in an Ultra Turrax equipment for 3 minutes, after which the suspension becomes thicker.

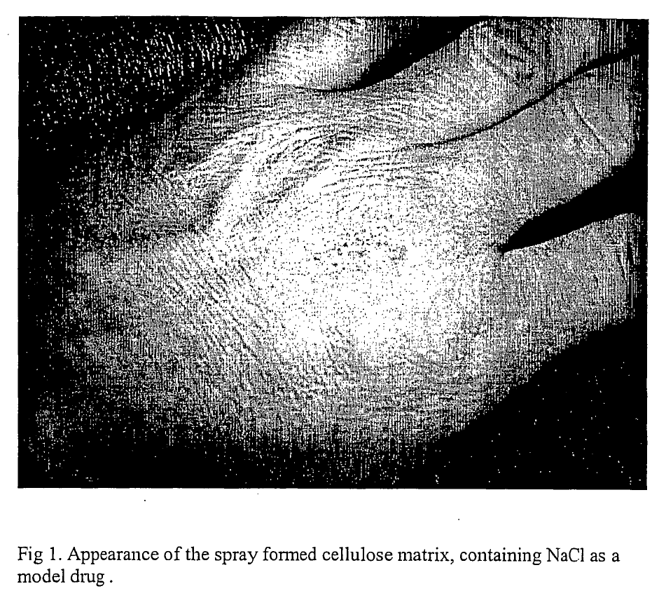

45 g of this suspension was placed into 100 ml Al-bottles which were sealed and pressurised by adding 15 g dimethylether. After spraying onto the skin, the water evaporated and left a continuous matrix of cellulose onto the skin that could not be shaken loos or wiped off with a dry napkin (FIG. 1). It was however easy to remove the cellulose layer with a wet napkin or by rinsing in water.

### Example 2

Spray Composition Containing Relatively Large Suspension Particles, Composed of Excipient Particles and Drug

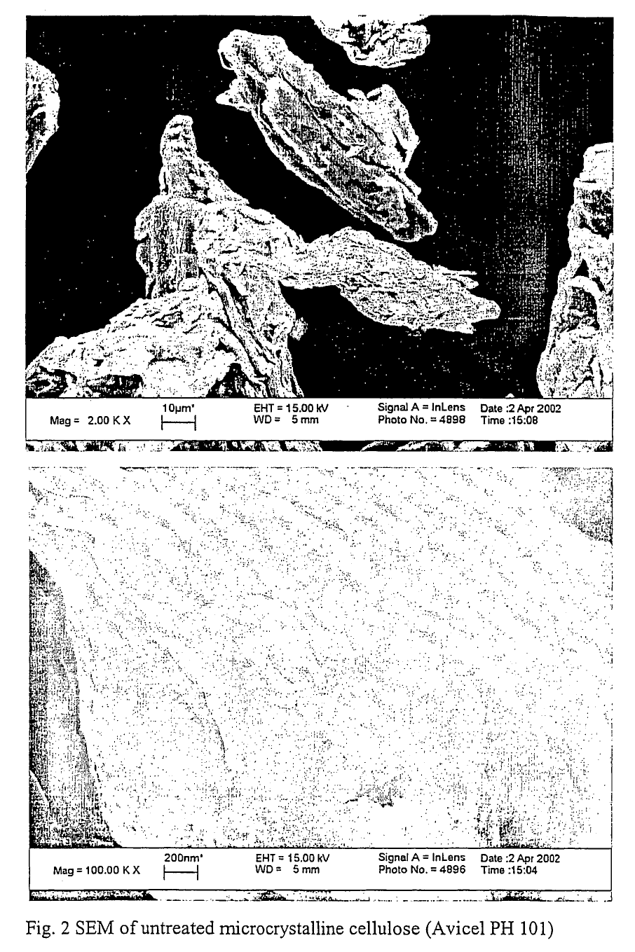

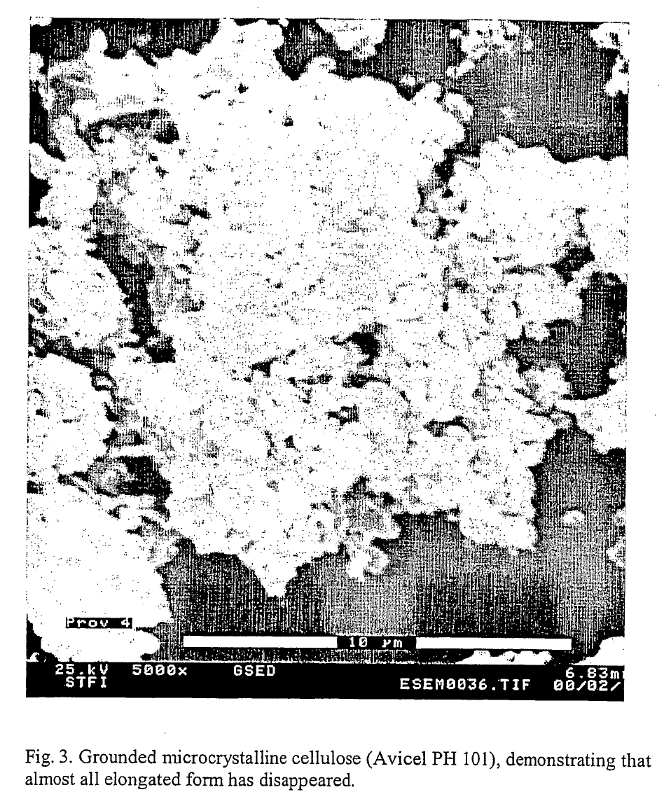

Microcrystalline cellulose (Avicel PH 101, FIG. 2) was grinded carefully (Retsch Model KMI, Retsch AG) with 1 part deionised water and 2 parts cellulose) for 2 hours. No reminding fibrous parts could be detected in microscope at 40× magnification, FIG. 3. Energy input about 4 kWh/kg or in the same order as when beating pulp for greaseproof paper manufacturing.

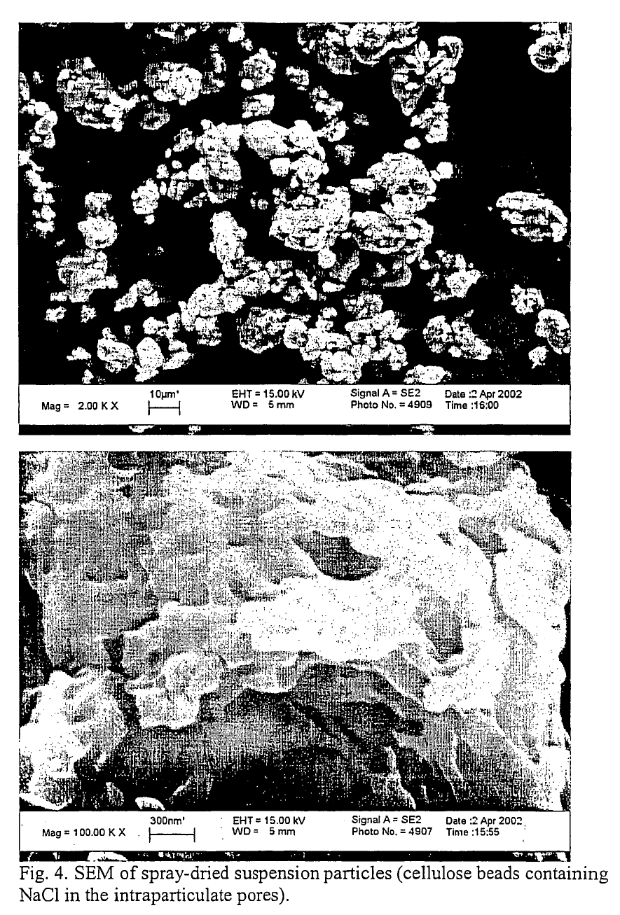

The grounded cellulose particles together with 0.2 g NaCl (used as a model drug substance) were suspended in water (10% dry solids) and spray-dried (Minor 53, Niro Atomizer AS, Denmark) at Tᵢₙ=210° C. and Tₒᵤₜ=95° C. with a feed-rate of 1.7 litre/h. The resulting particles are shown in FIG. 4.

The pressurised spray was made in the following way. First 13.5 g of the cellulose powder obtained from the spray-drier was added to 100 ml Al bottles. To this dry powder 31.5 g of water was added, the bottles sealed by crimping on a top valve and finally pressurised with 15 g dimethylether.

By spraying in a circle with Ø 15 cm in 3 s the surface was coverage with about 1.2 g dry solids. Repeated spraying onto napkins resulted in a standard deviation of about 5%. By shaking the napkin about 10% of the dry solids fell off.

### Example 3

Importance of Admixing Water to the Spray Liquid

With the purpose of showing the importance of water, present in the composition, Kleenex napkins was sprayed with a similar preparation as in example 2, but without water. The amount (%) that was adhered onto the napkin was measured. Without water the cellulose powder was dusting out into the room.

Another observation was that without water it was painful to spray cellulose onto the skin. A third observation is that the spray is non-flammable with water present.

| | | Amount adhering |
|---|---|---|
| | Amount adhering (%) | (%) Composition according |
| Spray | Composition without water | to example 2 Cellulose/ |
| time (s) | Cellulose/NaCl/dimethylether | NaCl/Water/dimethylether |
| 1 | 23 | — |
| 3 | 12 | 84 |
| 5 | 21 | 98 |
| 7 | 18 | 99 |

### Example 4

Reproducibility in Sprayed (Discharged) and Adhering Amount

Suspension particles were produced and loaded into spray bottles as described in example 2. The particles (size 45-106 μm) were sprayed onto a napkin from about a distance of 3 dm. The increase in weight was measured before drying, after drying and after drying and shaken the napkin for about 5 s. In the table below, the discharged amounts per second are given for various spraying times and the relative standard deviation in % is given within parenthesis.

| | Spray time (s) | | |
|---|---|---|---|
| | 1 | 2 | 3 |
| Total discharge (g/s) | 1.51 (8.44) | 1.39 (9.13) | 1.17 (8.74) |
| Dry solids discharge | 0.478 (10.40) | 0.445 (9.12) | 0.535 (10.1) |
| (g/s) | | | |
| Dry solids shaken loss | 4.6 | 8.2 | 8.6 |
| (%) | | | |

### Example 5

Effect of Drug (Ketoprofen) on the Particle Adhesion

Th drug ketoprofen is sparingly soluble in water. Therefore 75 g ketoprofen was dissolved in 450 g ethanol. The ethanol solution was added to the cellulose suspension (analogous with NaCl in Example 2) and the ketoprofen loaded particles were obtained by spray drying. The following mixture was spray dried; Ketoprofen/Ethanol/ground cellulose/water with the weight ratio (1/6/4.5/6.66. The resulting particles was filled into spray bottles, subsequently pressurised and the resulting spray was tested as described in Example 4, and compared with data from the composition of Example 2. The table below shows that the adhesion of the particles is increased when containing ketoprofen.

| | Suspension particles | |
|---|---|---|
| | Particles containing | Particles |
| | ketoprofen | containing NaCl |
| Total discharge [g/s] | 0.966 (57.98) | 1.51 (8.44) |
| Dry solids discharge (g/s) | 0.356 (21.1) | 0.478 (10.40) |
| Dry solids shaken loss (%) | 0.84 | 4.6 |

### Example 6

A Spray Preparation for Morphine for the In Situ Formation of a Drug Matrix on the Skin

In a study of Long⁵ the maximal daily dose of morphine given to patients is 198 mg. It is desired to be able to deliver that dose with a sustained release of morphine over at last 24 h to avoid disturbing the healing of the wound an also to avoid peaks in plasma concentration. In the following a calculation is given for a morphine spray.
⁵ Long T. D. Cathers T. A., Twillman R., O'Donnell T., Garrigues N. and Jones T., Morpine-Infused Silver Sulfadiazine (MISS) Cream for Burn Analgesia: A Pilot Study. J Burn Care & Rehabilitation 22 (2001) 118-123

It is assumed that the skin is injured so badly that the wound is oozing. When a cellulose layer is sprayed onto the skin (50 cm²) for 1 second, these layers becomes dry after about 10 minutes. This mans that about 0.6 g of water has evaporated with a speed of 83 mg water/h cm². Considering that the penetration resistance for absorption of morphine is so low, it is assumed that sink conditions is obtained on the skin surface. From experiments conducted in relation to Example 1 we know that we can apply about 3 g of matrix-forming cellulose particles by spraying 3-6 seconds onto a surface area of 154 cm². This will give a layer in the order of 100 μm.

The theoretical surface area available for diffusion is 154 cm² for a pure water solution, but the effective surface area taking part in drug release is proportional to the fraction of adhering particle surface area in contact with the skin, that means that a 50% cellulose suspension will have a surface area of approximately 77 cm² and that a “dry cellulose layer” containing somewhere around 10% moisture will have a diffusion area of 15.4 cm².

If the spray is formulated with morphine with a solubility of 1:5000⁶ (200 g/m³) and an estimation of a typical diffusion coefficient in the cellulose layer can be 10⁻¹⁰ m²/s, the following calculations can be made.
⁶ Therapeutic Drugs Dollary C. (Ed) Churchill Livingstone, Edingburgh (1991) p. M225

With the equation describing the released amount Q=AtDdc/dx

the released amount Q=188 mg morphine=0.188 g

surface area A=15.4 cm²=1.54 10⁻³ m²

Diffusion coefficient D=5*10⁻¹⁰ m²/s

Dc/dx=200/1 10⁻⁴ g/m³=2*10⁶ g/m

A typical release time can be calculated to be 34 hr.

This example demonstrates that by using a matrix, formed in situ, on the skin, an extended release preparation of sparingly soluble drugs can be obtained.

### Example 7

Spray Preparation Containing Relatively Large Suspension Particles, Composed of Excipient Particles and the Drug Ketoprofen

Chemicals

Ketoprofen, Batch: 052KL303, Sigma Chemie Gmbh, Germany.

Microcrystalline cellulose, Avicel PH 102, Lot:7505, FMC, Ireland.

Buffer pH 7.5, (PBS—Tween tablets Batch.1036TPT, Svanova Biotech AB, Sweden)

Aerosol Bottle

200 ml bottle of aluminium (inside covered with polyamid enamel, Cebal, France) with nozzle; 1x.0.24A Powder Shaft 3 mm ID 1,5) and push bottom; Ea Kosm. Apsl.020 F-3 mm (Deutsche Praäzisions-Ventil, Germany).

Making Ketoprofen/Cellulose Granules

The cellulose was grinded in a powder mill (Retsch KM 1, Germany) for 120 minutes. Before grinding the cellulose were wetted by addition of 50 g deionised water to 100 g cellulose. After grinding a suspension was made by adding 666 g of deionised water to the 100 g of grinded cellulose and the slurry was well stirred for 15 minutes with a hand mixer.

To the slurry a solution of ketoprofen was added, 100 grams of ketoprofen was dissolved in 600 grams of ethanol. The solution was than added to the microcrystalline cellulose slurry and was stirred (Hand mixer, Heidolph Diax 900, Germany) for another 10 minutes before being spray dried (Minor Type 53, Nitro Atomizer A.S, Denmark) with Tᵢₙ=205-210° C. and Tₒᵤₜ=95-100° C. The ketoprofen powder was seized and the fraction between 45-106 μm were used.

Filling Aerosol Bottle

Sodium chloride was mixed with ketoprofen/cellulose granules and the powder mixture was put into aerosol bottles. Deionised water was added, nozzles were put on and the bottles were sealed. The aerosols were vigorously shaken for 30 seconds to mix the content properly. Finally dimethyl ether was added in portions, with shaking between, through the nozzle until the final weight was achieved. The formulation is given in the table below.

| Substance | Function | Weight [g] |
|---|---|---|
| Ketoprofen^{a}/cellulose granules | Drug particles | 33.0 |
| Dimethylether | Propellant | 30.0 |
| Water | Vehicle | 63.0 |
| NaCl | Flocculation agent | 0.56 |

^{a}18% by weight ketoprofen in the granules

Release of Ketoprofen

To characterise drug release from matrixes of varying dimensions the suspension spray was applied on top of filter papers (Glass micro fibre filter GF/A, diameter; 150 mm, Whatman, Great Britain) resting on top of beakers filled with water. The spray was forced to pass through a 25 mm hole in a piece of wood fibreboard and different spraytimes were used to get different heights of the applied matrix. The weight of the applied spray on each filter paper was noticed. Filter papers were placed on top of a beaker filled up to the edge with 1175 ml of buffered water pH 7.5, 23° C. A soucer was put on top of each beaker to reduce the evaporation of buffer solution and a magnetic stirrer was used in the bottom to mix the release of ketoprofen properly. The release of ketoprofen was characterised by withdrawing samples as a function of time. The sample volume, 2.5 ml, was taken out from the dissolution beaker were replaced by the same volume of buffer solution so the total volume remained constant during the measurements. The concentration of ketoprofen in the sample was estimated with spectrophotometry (Hitachi U-1100, Japan), at 260 nm (Funk O et al 1993). After 7 days the filter paper was dumpt in the beaker to confirm that all the ketoprofen has been dissolved.

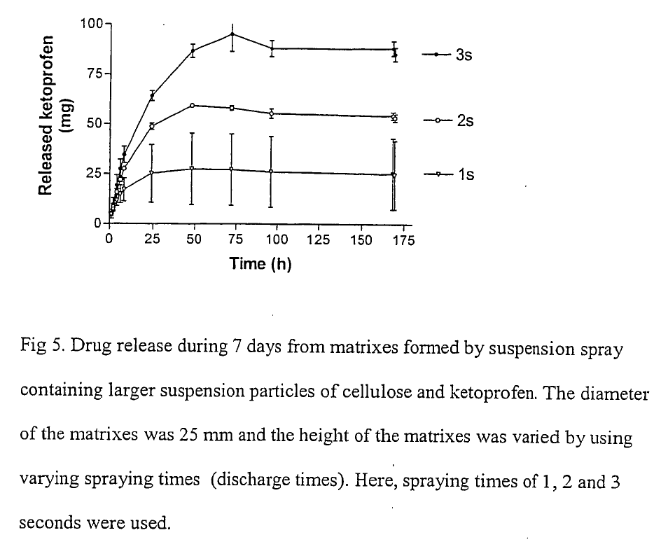

The results (FIG. 5) demonstrate that for all matrixes formed, the release of ketoprofen was significantly extended. For the matrix with lowest height (spraying for 1 second) the relese continued for approximately 24 hours, while matrixes formed after 2 second of spraying gave a doubling in release duration. Consequently, spraying for 3 seconds resulted in the thickest matrix and a release time of approximately 3 days. As expected the initial release rate was appromitaly the same (4 mg/hour) for all three systems and related only to the diameter of the matrix (25 mm) and independent on the matrix height.

### Example 8

Spray Preparation Containing Lidocaine Hydrochloride and Smaller Excipient Suspension Particles for Forming a Coherent, Porous Matrix, In Situ, on Skin and Comparison with a Conventional Gel Formulation

Spray preparations were produced and tested on drug release as described in Example 7, with the following modifications.

33 g of microcrystalline cellulose (Avicel PH 105) was placed (dry-filling) into 200 ml Al-bottles. 26 g of lidocaine hydrochloride was dissolved in 63 g of distilled water and the solution then filled into the Al-bottles. Sodium hydroxide was then added to adjust the pH to 7.5. The Al-bottles were then sealed and pressurised by adding 30 g dimethylether. The release of lidocaine hydrochloride was the monitored and compared with the release of lidocaine hydrochloride from a conventional gel formulation (Xylocain® 2% gel, AstraZeneca). The release experiments were conducted as described in Example 7. In these experiments the amount of preparations applied corresponded to an amount of approximately 80 mg lidocaine hydrochloride. The gel experiment was conducted as a single experiment (79.18 mg lidocaine hydrochloride), while the suspension spray experiment was conducted in triplicate (84.2, 92.7 and 80.0 mg of lidocaine hydrochloride, respectively).

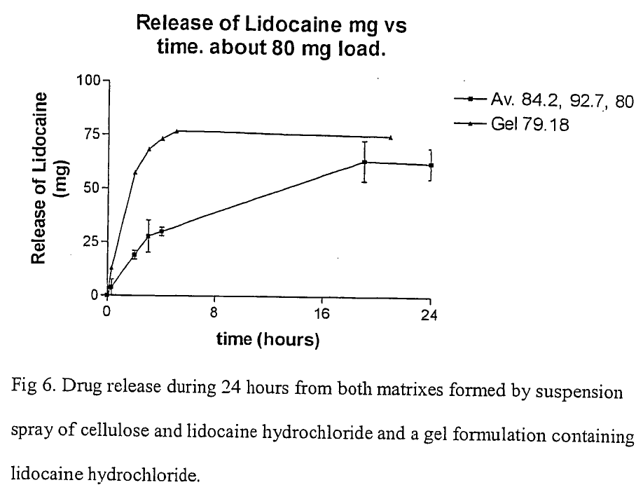

The results are presented in FIG. 6, were the drug release rate from a spray is compared with the release rate of lidocaine hydrochloride from a traditional gel formulation. It is evident that while the drug release from the gel is completed after approximately 4-6 hours, the drug release from the spray matrix is extended over a much longer time resulting in a release duration of approximately 20 hours.

While we have described a number of embodiments of the invention, it is obvious that this basic construction may be altered to generate other embodiments that utilise the methods described in this invention. Therefore, it will be appreciated the scope of this invention is defined by the claims appended here to rather than the specific embodiments which have been described by the examples.

1. A pharmaceutical composition, constituting a spray suspension comprising at least one liquid excipient and at least one solid excipient which essentially is insoluble in the liquid excipient, and at least one pharmaceutical active ingredient.

2. A pharmaceutical composition in accordance with claim 1, characterised in that the liquid excipient is a pressured aerosol propellant, such as dimethylether, butane, propane, mixtures of butane and propane, fluorinated hydro carbons, nitrogen, carbon dioxide and nitrous oxide.

3. A pharmaceutical composition in accordance with claim 2, characterised in that also water is included in the composition, preferably in a concentration between 10-95 w/w %, and more preferably in a concentration between 30-95%.

4. A pharmaceutical composition in accordance with claim 1, characterised in that the liquid excipient is water or a mixture of water and an organic solvent, such as alcohols.

5. A pharmaceutical composition in accordance with claim 1, characterised in that the solid excipent consists of inorganic salts or polymers selected from the group consisting of natural polymers, modified natural polymers, synthetic polymers and mixtures thereof.

6. A pharmaceutical composition in accordance with claim 5, characterised in that the polymeric material consist of natural polymers selected from the group consisting of native cellulose, such as Cellulose I.

7. A pharmaceutical composition in accordance with claim 6, characterised in that the native cellulose is micro crystalline cellulose or milled qualities of micro crystalline cellulose.

8. A pharmaceutical composition in accordance with claim 1, characterised in that the excipient particles are suspended in the liquid excipient, wherein the active ingredient is either dissolved, partly dissolved or suspended in the liquid or precipitated on the surface of the solid excipient and where the excipient particles after actuation can form a matrix, in-situ, on the administration site, such as the skin.

9. A pharmaceutical composition in accordance with claim 8, characterised in that the composition also contains at least one additional solid excipient which is capable of retarding the drug release from the matrix formed in-situ.

10. A pharmaceutical composition in accordance with claim 8, characterised in that at least 50% by weight of the excipient particles have a particle size not less than 0.1 pm and where at least 90% by weight of the excipient particles have a particle size less than 50 go.

11. A pharmaceutical composition in accordance with claim 1, characterised in that the excipient particles together with the active ingredient forms a plurality of larger individual particles (suspension particles).

12. A pharmaceutical composition in accordance with claim 11, characterised in that the excipient particles together with the active ingredient forms a plurality of larger individual particles that are porous and that the composition also contains at least one additional solid excipient which is capable of retarding the drug release from the suspension particles.

13. A pharmaceutical composition in accordance with claim 9, characterised in that the additional solid excipient is a polymer, with pronounced ductile properties thereby capable of reducing the porosity and/or average poor diameter of the suspension particles, or the matrix formed in-situ.

14. A pharmaceutical composition in accordance with claim 11, characterised in that the composition also contains at least one additional solid excipient which is capable of forming an outer membrane layer around the suspension particles, where the membrane layer retards the drug release and where the membrane layer is composed of non-polymeric- or polymeric materials such as calcium phosphate, ethyl cellulose, methacrylate copolymer, polyamide, polyethylene, polyvinyl alcohol or polyvinyl acetate.

15. A pharmaceutical composition in accordance with claim 11, characterised in that at least 50% by weight of the suspension particles have a particle size not less than 10 um and where at least 90% by weight have a particle size smaller than 150) J. m.

16. A pharmaceutical composition in accordance with claim 11, characterised in that the suspension particles have an essentially isodiametrical shape, and preferably the particles also have a smooth surface texture.

17. A method of preparing porous suspension particles comprising an active ingredient, in accordance with claim 11, characterised in that it comprises the steps of; a. wet-milling or dry-milling the solid excipient(s) or a mixture of at least one active ingredient and a solid excipient(s) in a milling equipment inducing essentially compression and shear forces, resulting in fine particulate quality, where more than 90% by weight is smaller than 5 Fm and preferably smaller than 2 um; and b. drying and aggregating the product of step a. or the product of step a. with the addition of at least one active ingredient, in fine particulate form, by e.g. spray-drying or any other drying procedure possible, which will produce essentially isodiametrical aggregate particles.

18. A method of preparing porous suspension particles comprising an active ingredient, characterised in that it comprises the steps of;
a. porous excipient particles, excluding any active ingredient, are prepared in accordance with the method described in claim 17; and
b. at least one active ingredient is added to the product of step a. whereby the active ingredient is essentially positioned within the pore structure of the product of step a.

19. A method of preparing non-porous suspension particles (including an active ingredient), in accordance with claim 14, characterised in that the active ingredient is applied, by e.g. a coating process, as an outer layer on solid, non-porous, excipient particles.

20. A method of applying a drug release retarding outer membrane layer to the suspension particles, prepared in accordance with the method described in claim 17, and where the membrane layer is composed of non-polymeric- or polymeric materials such as calcium phosphate, ethyl cellulose, methacrylate copolymer, polyamide, polyethylene, polyvinyl alcohol or polyvinyl acetate.

21. Suspension particles obtainable by a method according to claim 17.

22. A pharmaceutical preparation, utilising the composition in accordance with claim 1, characterised in that the preparation is a cutaneous spray, an ear spray or a nasal spray.

23. A pharmaceutical preparation, utilising the composition or the suspension particles in accordance with claim 22, characterised in that the preparation contains as the active substance, morphine, morphine sulphate, morphine hydrochloride, ketoprofen, lidocaine hydrochloride or other substances effective in the treatment of pain or capable of inducing anestethic effect.

24. A pharmaceutical preparation, utilising the composition in accordance with claim 22, characterised in that the preparation is in the form of a pressurised aerosol or mechanical pump device.

25. A method for treatment of disorders, wherein to an individual afflicted with disorder is administered a pharmaceutical composition, constituting a spray suspension comprising at least one liquid excipient and one solid excipient which essentially is insoluble in the liquid excipient and at least one pharmaceutical active ingredient.

26. A method for treatment of disorders according to claim 25 wherein the drug release rate is controlled by varying the area of said composition covering the skin of an individual.

27. A method for treatment of disorders according to claim 26 wherein the drug release rate is controlled by using a device with a range of increasingly sized openings or a device with a diaphragm where the opening diameter can be varied.

28. A method for treatment of disorders according to claim 25 wherein the drug release duration is controlled by varying the height of said composition covering the skin of an individual.

29. A method for treatment of disorders according to claim 28 wherein the drug release duration is controlled by using a specific spraying time.

30. A method for treatment of disorders according to claim 25 wherein the drug release rate is controlled by varying the area of said composition covering the skin of an individual, and wherein the drug release duration is controlled by varying the height of said composition covering the skin of an individual.

31. A method for treatment of disorders according to claim 30 wherein the drug release rate is controlled by using a device with a range of increasingly sized openings or a device with a diaphragm where the opening diameter can be varied.

32. A method for treatment of disorders according to claim 30 wherein the drug release duration is controlled by using a specific spraying time.
